(19) United States
(12) Patent Application Publication
(10) Pub. No.: US 2008/0100802 A1
(43) Pub. Date: May 1, 2008

(54) Ophthalmic measurement apparatus

(75) Inventor: Masaaki Hanebuchi, Nukata-gun (JP)

Correspondence Address: OLIFF & BERRIDGE, PLC, P.O. BOX 320850, ALEXANDRIA, VA, 22320-4850, US

(73) Assignee: NIDEK CO., LTD., Gamagori-shi (JP)

(21) Appl. No.: 11/976,685

(22) Filed: Oct. 26, 2007

(30) Foreign Application Priority Data
Oct. 31, 2006 (JP) 2006-296566

(51) Int. Cl. A61B 3/12

(52) U.S. Cl. 351/211

(57) ABSTRACT

An ophthalmic measurement apparatus which measures wavefront aberration of a patient's eye with high accuracy includes an irradiation optical system including a high-coherence measurement light source, a photo-receiving optical system including a light dividing element and a photodetector, a measurement light deflector placed in a position on an optical path of the irradiation optical system and not on that of the photo-receiving optical system, a memory storing deflection information and a pattern photo-received on the photodetector and associated with the information, and a control unit which drives the deflector to form patterns in different deflection states, controls the photodetector to photo-receive the patterns, reads out from the memory the information and the pattern, corrects the patterns based on their corresponding information by an amount of displacement of the read-out pattern with respect to a reference pattern, and performs addition to the corrected patterns so as to calculate the wavefront aberration.

### BACKGROUND OF THE INVENTION

1. Field of the Invention

The present invention relates to an ophthalmic measurement apparatus which measures wavefront aberration of a patient's eye.

2. Description of Related Art

Conventionally, there is known an apparatus which emits measurement light in a spot shape to a fundus of a patient's eye, photo-receives the measurement light reflected from the fundus on a photodetector that is a wavefront sensor, and measures wavefront aberration (especially, higher-order aberration) of a patient's eye (see U.S. Pat. No. 6,234,978 corresponding to Japanese Patent Application Unexamined Publication No. Hei 10-216092).

In the apparatus as described above, sometimes used is a measurement light source of high coherence (hereinafter, referred to simply as a high-coherence light source) such an SLD (super luminescence diode) and an LD (laser diode). The high-coherence light source is suitably used in improving measurement accuracy. However, speckle noise is detected by the photodetector. Accordingly, in order to further increase the measurement accuracy, it is preferable to suppress the speckle noise.

### SUMMARY OF THE INVENTION

An object of the invention is to provide an ophthalmic measurement apparatus which measures wavefront aberration of a patient's eye with high accuracy.

To achieve the objects and in accordance with the purpose of the present invention, an ophthalmic measurement apparatus includes a measurement light irradiation optical system including a high-coherence measurement light source, for irradiating a fundus of the patient's eye with measurement light in a spot shape which is emitted from the measurement light source, a photo-receiving optical system including a light dividing element which divides the measurement light reflected from the fundus into a plurality of light bundles and a photodetector which photo-receives the divided light bundles as a pattern, a deflector which deflects the measurement light with which the fundus is to be irradiated and is placed in a position which is on an optical path of the measurement light irradiation optical system and is not on an optical path of the photo-receiving optical system, a memory which stores deflection information that specifies a state of deflection of the measurement light which is made by the deflector and a pattern of the light bundles which is photo-received on the photodetector, the pattern being associated with the deflection information, and a control unit which drives the deflector to change the deflection state of the measurement light so as to form patterns in different deflection states, controls the photodetector to photo-receive the patterns in the different deflection states, reads out from the memory the deflection information and the pattern associated therewith, corrects the patterns in the different deflection states based on their corresponding deflection information by an amount of displacement of the read-out pattern with respect to a reference pattern, and performs addition to the corrected patterns in the different deflection states so as to calculate the wavefront aberration.

Additional objects and advantages of the invention are set forth in the description which follows, are obvious from the description, or may be learned by practicing the invention. The objects and advantages of the invention may be realized and attained by the ophthalmic measurement apparatus in the claims.

### BRIEF DESCRIPTION OF THE DRAWINGS

The accompanying drawings, which are incorporated in and constitute a part of this specification, illustrate embodiments of the present invention and, together with the description, serve to explain the objects, advantages and principles of the invention. In the drawings,

FIG. 1 is a view showing an optical system and a control system of an ophthalmic measurement apparatus according to a preferred embodiment of the present invention; and

FIG. 2 is a view for illustrating displacement of a pattern when the position of measurement light on a fundus is changed by a deflector and correction to the pattern.

### DETAILED DESCRIPTION OF THE PREFERRED EMBODIMENTS

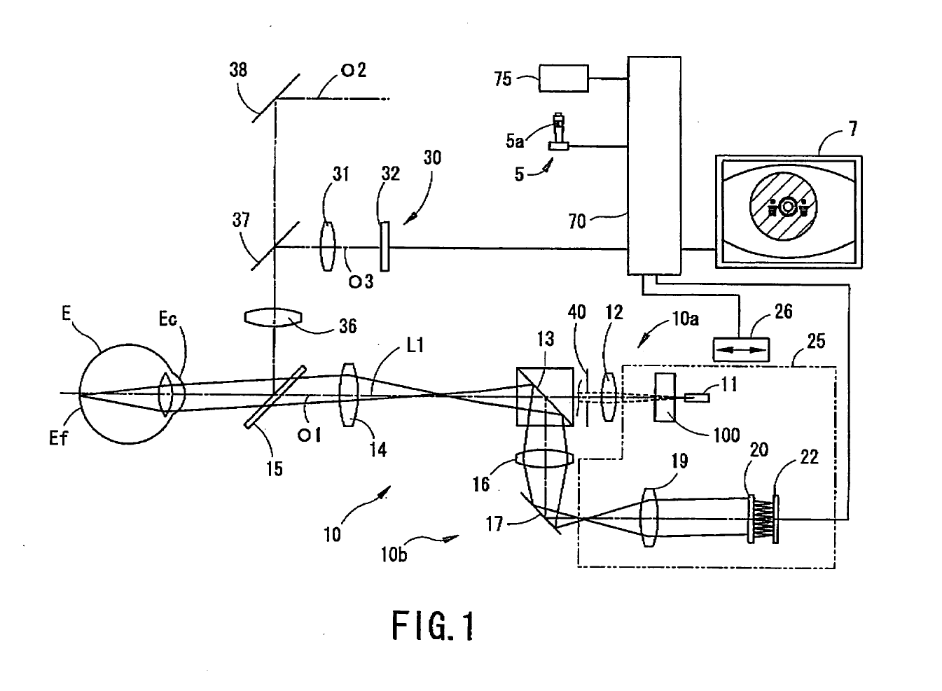

A description of one preferred embodiment of an ophthalmic measurement apparatus embodied by the present invention is provided below with reference to the accompanying drawings. FIG. 1 is a view showing an optical system and a control system of the ophthalmic measurement apparatus according to the preferred embodiment of the present invention. A dichroic mirror 15 is placed in front of a patient's eye E. On a transmission optical path O1 of the mirror 15, a wavefront aberration measurement optical system 10 for measuring wavefront aberration of the patient's eye E is placed. The measurement optical system 10 includes a measurement light irradiation optical system 10a for irradiating a fundus Ef with measurement light in a spot shape emitted from a measurement light source 11, and a photo-receiving optical system 10b for dividing the measurement light (reflection light) reflected from the fundus Ef into a plurality of light bundles so as to photo-receive the light bundles as a pattern of a plurality of target images (dot images) on a two-dimensional photodetector 22. Based on output from the two-dimensional photodetector 22, wavefront aberration of the patient's eye E is measured.

The measurement light irradiation optical system 10a includes the measurement light source 11, a light deflecting member 100 that defines a deflector which deflects the measurement light, a relay lens 12, a diaphragm 40, an objective lens 14, which are placed in this order in a direction from the measurement light source 11 to the patient's eye E. For the measurement light source 11, a high intensity and high coherence light source having a small light source portion such an SLD and an LD is used. The measurement light source 11 is placed in a position conjugate with the fundus Ff. The measurement light may be emitted from an SLD light source via an optical fiber, in which case an output terminal of the optical fiber is regarded as the measurement light source 11. The diaphragm 40 plays a role in reducing the diameter of the measurement light to be projected onto the fundus Ef so as to form a sharp spot image on the fundus Ef. The diaphragm 40 is placed on an optical path of the measurement light irradiation optical system 10a (further, it is preferable that the diaphragm 40 is placed in a position conjugate with a cornea Ec).

In addition, the light deflecting member 100 deflects the measurement light to be projected onto the fundus Ef in a direction perpendicular to a measurement optical axis L1. The light deflecting member 100 is placed in a position which is on the optical path of the measurement light irradiation optical system 10a and is not on an optical path of the photo-receiving optical system 10b (e.g., a position between the measurement light source 11 and the relay lens 12) In the preferred embodiment of the present invention, an acoustooptical deflector which non-mechanically deflects light is used as the light deflecting member 100, which is not limited thereto. For the light deflecting member 100, a light deflection prism or a movable reflection mirror may be used.

The photo-receiving optical system 10b includes the objective lens 14, a half mirror 13, a relay lens 16, a total reflection mirror 17, a collimator lens 19, a microlens array 20, and the two-dimensional photodetector 22 which photo-receives the light bundles passing through the array 20, which are placed in this order from the front of the patient's eye E. The half mirror 13 transmits the measurement light from the light source 11. In addition, the half mirror 13 reflects the reflection light from the fundus Ef. The photo-receiving optical system 10b is arranged such that a pupil of the patient's eye E and the array 20 have an optically conjugate relationship approximately. The microlens array 20 includes microlenses which are arranged two-dimensionally on a surface perpendicular to the measurement optical axis L1, and a light shielding plate. The array 20 divides the reflection light from the fundus Ef into the plurality of light bundles (i.e., the array 20 acts as a light dividing element). Incidentally, the photo-receiving optical system 10b in the preferred embodiment of the present invention is configured as a Shack-Hartmann wavefront sensor. Meanwhile, the photo-receiving optical system 10b may be configured as a Talbot wavefront sensor such that an orthogonal grid mask is placed in a position conjugate with a pupil so as to photo-receive light transmitted through the mask on a two-dimensional photodetector (see Japanese Patent Application Unexamined Publication No. 2006-149871).

In addition, in the preferred embodiment of the present invention, the measurement light source 11, the collimator lens 19, the array 20 and the two-dimensional photodetector 22 are moved integrally as a unit 25 in a direction of the optical axis L1 by a moving mechanism 26. In this case, the unit 25 is moved in the optical axis L1 direction so that the measurement light source 11 and the two-dimensional photodetector 22 have an optically conjugate relationship with the fundus Ef in accordance with a spherical refractive error of the patient's eye E. In other words, the unit 25 functions as a vision correcting mechanism for correcting the spherical refractive error of the patient's eye E.

Meanwhile, in a reflecting direction of the mirror 15, an objective lens 36 which is used for observing the patient's eye E, a dichroic mirror 37 and a tot al reflection mirror 38 are placed. On an optical path O2 in a reflecting direction of the mirror 38, a fixation target projection optical system for making the patient's eye E fixate on a fixation target is placed (not illustrated).

On an optical path O3 in a reflecting direction of the dichroic mirror 37, an observation optical system 30 for photographing the patient's eye E so as to obtain an image thereof is placed. The observation optical system 30 includes an image forming lens 31, and a two-dimensional image-pickup element 32 such as an area CCD (charge-coupled device) which is placed in a position approximately conjugate with the vicinity of an anterior-segment of the patient's eye E.

Incidentally, the dichroic mirror 15 has a property of transmitting light emitted from the measurement light source 11 and reflecting light (near infrared light) emitted from a light source for anterior-segment illumination (not illustrated) and a light source for alignment (not illustrated) and visible light. The dichroic mirror 37 has a property of transmitting the visible light and reflecting the near infrared light.

The light emitted from the light source for anterior-segment illumination and reflected from the anterior-segment forms an image of the anterior-segment on the two-dimensional image-pickup element 32 via the dichroic mirror 15, the objective lens 36, the dichroic mirror 37 and the image forming lens 31. The light from the fixation target projection optical system (not illustrated) is reflected by the mirror 38, travels on an optical path in the reverse direction to the direction in which the above-described anterior-segment reflection light travels on the optical path, and reaches the fundus Ef.

A control part 70 is programmed to obtain an output image signal from the two-dimensional photodetector 22 and analyze the wavefront aberration of the patient's eye E. Further, the control part 70 analyzes optical characteristics of the patient's eye E. The control part 70 is connected with the light source 11, the two-dimensional photodetector 22, the light deflecting member 100, a memory 75, the moving mechanism 26, the two-dimensional image-pickup element 32, a display monitor 7 which displays the image of the anterior-segment and a measurement result, and a joystick 5.

On a display screen of the monitor 7, the image of the anterior-segment which is picked up by the two-dimensional image-pickup element 32 is displayed. An examiner moves an apparatus cabinet which houses the entire optical system with the use of the joystick 5 while watching the monitor 7. In this manner, the examiner performs alignment of the measurement optical axis L1 with respect to the patient's eye E. After the alignment, a measurement starting switch 5a mounted at the tip of the joystick 5 is pushed to generate a trigger signal for measurement. Then, the control part 70 controls the measurement light source 11 to light up and starts measurement based on the trigger signal.

The measurement light emitted from the measurement light source 11 is projected onto the fundus Ef via the light deflecting member 100, the relay lens 12, the diaphragm 40, the half mirror 13, the objective lens 14, the dichroic mirror 15, and the pupil of the patient's eye E. Thus, a point-light-source image is formed on the fundus Ef of the patient's eye E.

At this time, when the light deflecting member 100 is driven by the control part 70, the measurement light which passes through the light deflecting member 100 is deflected in the direction perpendicular to the measurement optical axis L1, and the point-light-source image is two-dimensionally moved on the fundus Ef of the patient's eye E. Accordingly, the position of the measurement light with which the fundus Ef is irradiated is arranged to be changeable in chronological order. Besides, the light deflecting member 100 is arranged to deflect the measurement light in each of right, left, up and down directions by a given distance with respect to the measurement optical axis L1 in chronological order. In the apparatus according to the present invention, items of deflection information on the measurement light deflected by the light deflecting member 100 are predetermined per measurement of the optical characteristics of the patient's eye E (e.g., wavefront aberration distribution and eye refractive power distribution). The deflection information including a deflection direction, a deflection amount and the number of times of deflection is stored in advance in the memory 75. At the time of the measurement, the control part 70 drives the light deflecting member 100 based on the deflection information so as to deflect the measurement light a predetermined number of times.

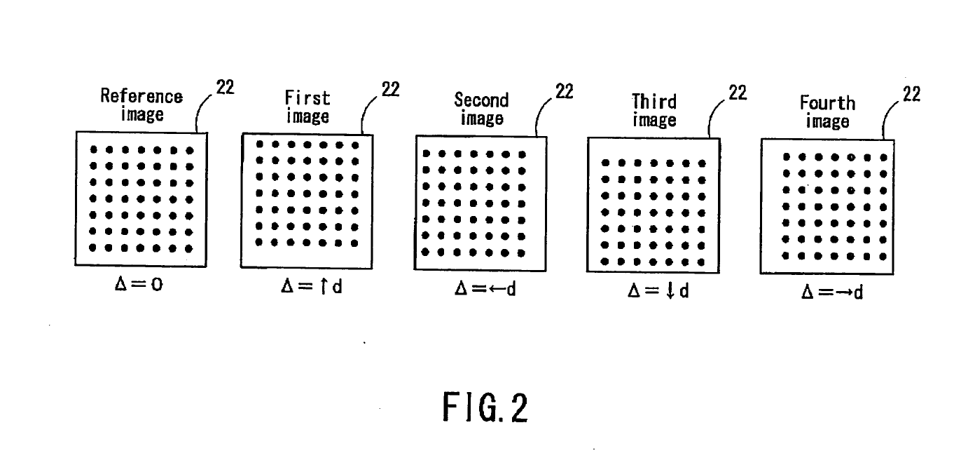

The measurement light which is projected onto the fundus Ef so as to form the point-light-source image thereon is reflected from the fundus Ef, and the reflection light is emitted from the patient's eye E and is transmitted through the dichroic mirror 15 so as to be collected by the objective lens 14. Then, the reflection light is reflected by the half mirror 13, and is reflected by the total reflection mirror 17 after being collected once by the relay lens 16. The light reflected by the total reflection mirror 17 passes through the collimator lens 19 and is divided into the plurality of light bundles by the microlens array 20 so as to be photo-received on the two-dimensional photodetector 22. Incidentally, by deflecting the measurement light as described above, the pattern photo-received on the two-dimensional photodetector 22 is entirely displaced as shown in FIG. 2 in accordance with the deflection direction of the measurement light.

The pattern of the light bundles divided into which by the microlens array 20 and photo-received on the two-dimensional photodetector 22 (see FIG. 2) varies with the degree of the aberration (low-order aberration and higher-order aberration) of the patient's eye E. Accordingly, by analyzing a pattern which is formed by the reflection light from the patient's eye E with respect to a pattern which is formed by aberration-free light, the wavefront aberration distribution and the eye refractive power distribution of the patient's eye E can be measured.

Hereinafter, referring to FIG. 2, a description of a technique to obtain a pattern every time the position of the measurement light is changed by the light deflecting member 100, to correct the obtained patterns on the photo-received positions based on their corresponding deflection information, to subject the corrected patterns to adding processing so as to obtain the wavefront aberration of the patient's eye E will be provided.

The control part 70 obtains a plurality of times the pattern which is detected by the two-dimensional photodetector 22 (picks up pattern images). Then, the control part 70 stores the respectively-obtained pattern images as image data in the memory 75, which are used for the adding processing. The obtainment of the pattern is performed at predetermined time intervals (e.g., at intervals of 1/30 second), and the obtained image data are successively output to the memory 75. In the preferred embodiment of the present invention, the obtained pattern images are referred to as a first image, a second image, a third image and a fourth image in order of the obtainment.

In addition, the control part 70 drives the light deflecting member 100 to change a deflection state (the deflection direction and the deflection amount) of the measurement light every time one frame of the pattern image is obtained as described above. The first image shows the pattern obtained when the measurement light is deflected upward with respect to the measurement optical axis L1. The second image shows the pattern obtained when the measurement light is deflected leftward with respect to the measurement optical axis L1. The third image shows the pattern obtained when the measurement light is deflected downward with respect to the measurement optical axis L1. The fourth image shows the pattern obtained when the measurement light is deflected rightward with respect to the measurement optical axis L1. As above, the pattern images are respectively associated with the deflection states of the measurement light. Incidentally, a reference image (reference pattern) in FIG. 2 shows the pattern obtained when the measurement light is coaxial with the measurement optical axis L1 on the fundus (when the measurement light is not deflected).

Since the position of the point-light-source image on the fundus Ef differs according to the deflection state of the measurement light deflected by the light deflecting member 100, the photo-received positions of the patterns on the two-dimensional photodetector 22 are different from each other. A positional relationship (spaces) between the dot images docs not vary. The pattern is displaced entirely in a predetermined direction by a predetermined amount. In the first image, the pattern is displaced upward by Δd with respect to the reference image. In the second image, the pattern is displaced leftward by Δd with respect to the reference image. In the third image, the pattern is displaced downward by Δd with respect to the reference image. In the fourth image, the pattern is displaced rightward by Δd with respect to the reference image.

Next, the control part 70 shifts to arithmetic processing in order to obtain the wavefront aberration. The control part 70 corrects, based on the deflection information stored in advance in the memory 75, the patterns on the photo-received positions for the displacements which result from the deflection of the measurement light made by the light deflecting member 100. To be more specific, data on displacements of photo-received positions of patterns of deflected measurement light with respect to a photo-received position of a pattern of not-deflected measurement light are stored in advance in the memory 75, and the control part 70 makes correction processing on the patterns on the photo-received positions using their corresponding displacement data. For example, in the first image in FIG. 2, the pattern is offset so as to compensate for the upward displacement Δ↑d. In the second image in FIG. 2, the pattern is offset so as to compensate for the leftward displacement Δ←d.

Next, the control part 70 subjects the corrected image data to the adding processing. Through the adding processing, intensity values of the dot images are increased. Accordingly, contrast between the respective dot images and a noise component is made clear, improving accuracy in positional detection of the dot images.

Thereafter, the control part-70 detects deviation amounts of the respective dot images in the pattern subjected to the adding processing. Based on the detected deviation amounts, the control part 70 obtains the inclination of a wave front of the reflection light. In order to analyze the wave front inclination, a known mathematical technique can be employed (see U.S. Pat. No. 5,963,300 corresponding to a published Japanese translation of PCT international publication for patent applications, No. 2003-526404). The analyzed wave front inclination is quantified by applying expansion of the known Zernike's polynomial, A spherical refractive error(S), an astigmatic refractive error (C), and an astigmatic axial angle (A) are expressed in terms of polynomials of degree two or lower. In addition, higher-order aberrations are expressed in terms of polynomials of degree three or higher. A result of the analysis of the optical characteristics including the wavefront aberration distribution and the eye refractive power distribution is displayed in the form of a map on the monitor 7 connected to the control part 70.

As described above, the measurement light is deflected by the light deflecting member 100 at the time of obtaining the patterns. Accordingly, speckle noise which is detected together with the patterns by the two-dimensional photodetector 22 is changed in accordance with the deflection state of the measurement light. By subjecting the patterns including the speckle noise to the adding processing, the speckle noise is relatively cancelled out and eliminated.

In addition, in the preferred embodiment of the present invention, the light deflecting member 100 which deflects the measurement light is placed in the position which is on the optical path of the measurement light irradiation optical system 10a and is not on the optical path of the photo-receiving optical system 10b. By this placement, the measurement light deflected by the light deflecting member 100 has a small diameter, being hardly influenced by the aberration. Accordingly, a pattern in which the aberration is suppressed can be obtained. Besides, the position of the light deflecting member 100 is not limited in particular if the position is on the optical path of the measurement light irradiation optical system 10a and is not on the optical path of the photo-receiving optical system 10b. Accordingly, optical design of this apparatus is facilitated. Incidentally, in consideration of the deflection amount, it is preferable for the light deflecting member 100 to be placed in a position apart from the light source 11.

In contrast, if the light deflecting member 100 is placed on the optical path common to the measurement light irradiation optical system 10a and the photo-receiving optical system 10b, the light deflecting member 100 needs to be placed in a position conjugate with the pupil. By this placement, the measurement light deflected by the light deflecting member 100 has a large diameter, being inevitably influenced by the aberration. Due to this, the measurement accuracy is decreased (especially in a peripheral portion of the measurement light). In addition, if a reflection surface of a movable mirror or one surface of a prism of the apparatus is arranged to be a spherical in order to prevent the influence of the aberration, high-priced optical members are to be used, leading to increases in the cost of the apparatus.

Incidentally, in the above description, the apparatus is configured to deflect the light to be projected onto the fundus Ef with the use of the light deflecting member 100, which is not limited thereto. For example, the apparatus may be configured to deflect the light to be projected onto the fundus Ef by mechanically moving the measurement light source 11 or the relay lens 12.

The foregoing description of the preferred embodiments of the invention has been presented for purposes of illustration and description. It is not intended to be exhaustive or to limit the invention to the precise form disclosed, and modifications and variations are possible in the light of the above teachings or may be acquired from practice of the invention. The embodiments chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. An ophthalmic measurement apparatus which measures wavefront aberration of a patient's eye, the apparatus comprising:
a measurement light irradiation optical system including a high-coherence measurement light source, for irradiating a fundus of the patient's eye with measurement light in a spot shape which is emitted from the measurement light source;
a photo-receiving optical system including:
a light dividing element which divides the measurement light reflected from the fundus into a plurality of light bundles; and
a photodetector which photo-receives the divided light bundles as a pattern;
a deflector which deflects the measurement light with which the fundus is to be irradiated, and is placed in a position which is on an optical path of the measurement light irradiation optical system and is not on an optical path of the photo-receiving optical system;
a memory which stores:
deflection information that specifies a state of deflection of the measurement light which is made by the deflector; and
a pattern of the light bundles which is photo-received on the photodetector, the pattern being associated with the deflection information; and
a control unit which drives the deflector to change the deflection state of the measurement light so as to form patterns in different deflection states, controls the photodetector to photo-receive the patterns in the different deflection states, reads out from the memory the deflection information and the pattern associated therewith, corrects the patterns in the different deflection states based on their corresponding deflection information by an amount of displacement of the read-out pattern with respect to a reference pattern, and performs addition to the corrected patterns in the different deflection states so as to calculate the wavefront aberration.

2. The ophthalmic measurement apparatus according to claim 1, wherein the reference pattern is a pattern which is photo-received on the photodetector when the measurement light is not deflected by the deflector.

3. The ophthalmic measurement apparatus according to claim 1, wherein the deflector is an acoustooptical deflector.
